(19) United States
(12) Patent Application Publication
(10) Pub. No.: US 2022/0304939 A1
(43) Pub. Date: Sep. 29, 2022

(54) ANTIMICROBIAL WIPES

(71) Applicant: 3M INNOVATIVE PROPERTIES COMPANY, St. Paul, MN (US)

(72) Inventors: Ranjani V. PARTHASARATHY, Woodbury, MN (US); Max A. KRUZIKI, New Brighton, MN (US); Joseph A. DUNBAR, Woodbury, MN (US)

(21) Appl. No.: 17/618,983

(22) PCT Filed: Jun. 18, 2020
(86) PCT No.: PCT/IB2020/055741

§ 371 Date: Dec. 14, 2021

Related U.S. Application Data
(60) Provisional application No. 62/863,966, filed on Jun. 20, 2019.

(51) Int. Cl. A61K 9/70; A61K 31/155; A61P 31/02; A61P 17/02

(52) U.S. Cl. CPC A61K 9/70; A61K 31/155; A61P 31/02; A61P 17/02

(57) ABSTRACT

A pre-loaded wipe, including a plurality of fibers having a denier value of less than about 1.0, and having a cationic antimicrobial composition distributed throughout the plurality of fibers. The cationic antimicrobial composition includes a cationic antiseptic compound chosen from bispyridines, biguanides, bisbiguanides, polymeric biguanides, salts thereof, and mixtures and combinations thereof. The pre-loaded wipe has good properties for releasing the cationic antiseptic compound when wiped on a treatment site.

### BACKGROUND

Routine pre-operative cleansing of the skin, mucosae, wound or surgical site with an antiseptic is important to prepare a patient for a surgical procedure. The purpose of preoperative skin antisepsis is to reduce the bioburden of microorganisms on the skin and thus reduce the risk of inoculation of the surgical site with potentially infecting organisms that reside on the skin.

Bacterial bioburden should be maintained as low as possible prior to making an incision, during surgery, and after closing the incision to reduce the rate of surgical site infections. Some common preoperative skin preparations include lower monohydric alcohols such as, for example, isopropyl alcohol (IPA), in combination with antiseptic compounds such as chlorhexidine, and iodine/iodophors. These preoperative skin preparations are fast-acting antiseptics (due to the alcohol) with persistent activity (due to the chlorhexidine or iodophor). These antiseptic compounds are effective against gram-positive and gram-negative bacteria, fungi and most viruses, and kill microbes by destroying the cell membrane. The antiseptic compounds also bind to the surface of the skin and can be released over time, which can provide persistent antimicrobial activity.

The antiseptic agent is often applied with a wipe or washcloth. However, in some instances the wipe material is made of fibers having a high surface area, which can result in absorption of the antiseptic agent into the fibers and reducing release onto the skin. Additionally, the fibers can pick up dirt, dust, or bodily fluids, such that release of the antiseptic agent may be hindered. There is a need for wipes loaded with antiseptic agent that can more efficiently deliver antiseptic agents.

### SUMMARY

The disclosed wipe is loaded with an antiseptic agent and is effective at delivering the antiseptic agent to a contact surface. In instances when the contact surface is porous, like skin, hair, mucosae, wounds, and surgical sites, the disclosed wipe loaded with an antiseptic agent is effective at delivering antiseptic agent into the porous surface and removing contaminants from that surface.

In one aspect, the present disclosure is directed to a wipe having a plurality of fibers with a denier value of less than about 1.0. The wipe is pre-loaded with a cationic antimicrobial composition distributed throughout the plurality of fibers. The cationic antimicrobial composition includes a cationic antiseptic compound chosen from bispyridines, biguanides, bisbiguanides, polymeric biguanides, salts thereof, and mixtures and combinations thereof. In some embodiments, the cationic antiseptic compound is chosen from octenidine dihydrochloride, polyhexamethylene biguanide (PHMB), chlorhexidine gluconate (CHG), salts thereof, and combinations thereof.

In some embodiments, the cationic antimicrobial composition in the pre-loaded wipe includes a surfactant system chosen from a first surfactant having a Hydrophile-Lipophile Balance (“HLB”) value of less than about 10, a second surfactant different from the first surfactant and having an HLB value in a range from about 10 to about 14, a third surfactant different from both the first surfactant and the second surfactant having an HLB value in a range from about 14 to about 18, and combinations thereof.

In some embodiments, the cationic antimicrobial composition in the pre-loaded wipe includes a surfactant chosen from a fatty acid monoester or a polyethylene glycol (“PEG”) fatty acid ester having an HLB value of less than about 10.

In some embodiments, the cationic antimicrobial composition in the pre-loaded wipe includes a surfactant that includes a PEG compound with an HLB value of greater than about 8 and less than about 14.

In some embodiments, the cationic antimicrobial composition in the pre-loaded wipe includes a surfactant chosen from a surfactant having an HLB value between 8 and 14.

In another aspect, the present disclosure is directed to a system of disinfecting a treatment site. The treatment site is chosen from skin, hair, mucosae, wounds, and body cavities. The system includes a cationic antimicrobial composition distributed throughout a wipe. The wipe has a plurality of fibers having a denier value of less than about 1.0. The cationic antimicrobial composition is distributed from the wipe to the treatment site, in an amount effective to kill a microorganism chosen from bacteria, fungi, viruses, and mixtures and combinations thereof.

In some embodiments of the system, the cationic antimicrobial composition includes a surfactant system chosen from a first surfactant having an HLB value of less than about 10, a second surfactant different from the first surfactant and having an HLB value in a range from about 10 to about 14, a third surfactant different from both the first surfactant and the second surfactant having an HLB value in a range from about 14 to about 18, and combinations thereof.

In another aspect, the present disclosure is directed to a method of disinfecting a treatment site chosen from skin, hair, mucosae, wounds, and body cavities. The method includes distributing a cationic antimicrobial composition from a pre-loaded wipe to the treatment site, the pre-loaded wipe including the cationic antimicrobial composition distributed throughout a plurality of fibers having a denier value of less than about 1.0. The cationic antiseptic compound is distributed from the pre-loaded wipe to the treatment site in an amount effective to kill a microorganism chosen from bacteria, fungi, viruses, and mixtures and combinations thereof.

In some embodiments of the method, the cationic antimicrobial composition includes a surfactant system chosen from a first surfactant having an HLB value of less than about 10, a second surfactant different from the first surfactant and having an HLB value in a range from about 10 to about 14, a third surfactant different from both the first surfactant and the second surfactant having an HLB value in a range from about 14 to about 18, and combinations thereof.

The details of one or more embodiments of the invention are set forth in the accompanying drawing and the description below. Other features, objects, and advantages of the invention will be apparent from the description and from the claims.

### BRIEF DESCRIPTION OF THE DRAWINGS

FIG. 1 is a schematic diagram of a pre-loaded wipe of the present disclosure that can be used to apply the antimicrobial compositions of the present disclosure to a treatment site.

FIG. 2 is a schematic diagram of a mitt-shaped pre-loaded wipe of the present disclosure that can be used to apply the antimicrobial compositions of the present disclosure to a treatment site.

### DETAILED DESCRIPTION

In one aspect, the present disclosure is directed to pre-loaded wipes that include fibers and a cationic antimicrobial composition that can be used to disinfect a wide variety of treatment sites such as, for example, skin, hair, mucosae, wounds, and surgical sites. The wipes have a plurality of fibers with a denier value of less than about 1.0. The wipes are pre-loaded with a cationic antimicrobial composition distributed throughout the plurality of fibers. The cationic antimicrobial composition can be distributed from the pre-loaded wipes to treatment sites and surfaces in an amount effective to kill microorganisms chosen from bacteria, fungi, viruses, and mixtures and combinations thereof.

The cationic antimicrobial composition includes at least one cationic antiseptic compound. Suitable cationic antiseptic compounds include bispyridines, biguanides, bisbiguanides, polymeric biguanides, salts thereof, and mixtures and combinations thereof.

In some embodiments, the cationic antiseptic compound is chosen from polyhexamethylene biguanide (PHMB), chlorhexidine, octenidine, salts thereof, and mixtures and combinations thereof. In some embodiments, the chlorhexidine is a soluble salt, and the diacetate and digluconate salts have been found to be particularly useful in the cationic antimicrobial composition. In various embodiments, octenidine could be in the form of the dihydrochloride or other suitable salts such as gluconate, citrate, lactate, and the like. In some embodiments, the cationic antiseptic compound includes chlorhexidine gluconate (CHG), also referred to as chlorhexidine digluconate, or consists of CHG. CHG is a cationic antiseptic that is effective on both gram-positive and gram-negative bacteria. CHG is both bacteriocidal (kills) and bacteriostatic (stops reproductions) of any bacteria on mammalian skin.

In some embodiments, which are not intended to be limiting, the cationic antiseptic compound is present in the cationic antimicrobial composition at about 0.05 wt. % to about 10 wt. %, or about 0.1% wt. % to about 5 wt. %, or about 0.1 to 0.4% or about 1 wt. % to about 3 wt. %, or about 1.5 wt. % to about 2.5 wt. %, based on the total weight of the composition (±1%).

In some embodiments, which are not intended to be limiting, the cationic antiseptic compound is released from the pre-loaded wipe to a treatment site in an amount of at least 50 wt. %, or at least 60 wt. %, or at least 70 wt. %, or at least 80 wt. %, or at least 90 wt. %, based on the total weight of cationic antiseptic compound in the antimicrobial solution.

In some embodiment, it may be desirable to further include a surfactant system with the cationic antiseptic that is pre-loaded in the wipe. A surfactant system can further enable release of the cationic antiseptic from a pre-loaded wipe with fibers having denier less than 1, such as microfibers or nanofibers. The combination of cationic antiseptic and surfactant can allow for improved access to bacteria that are not accessible on the surface of a treatment site. It is thought that the combination of cationic antiseptic and surfactant reduces shear forces at the interface between the pre-loaded wipe and the treatment site, allowing for improved dislodgement of microorganisms, and better mechanical forces at the interface.

The surfactants in the present disclosure are described as having a Hydrophile-Lipophile Balance (“HLB”) value. The HLB value is used in the present application as an empirical expression for the relationship of the hydrophilic and hydrophobic groups of the surfactant in the composition. In most cases the higher the HLB value, the more water-soluble the surfactant. The selected range of HLB values may vary depending on other additives in the cationic antimicrobial composition.

In the present disclosure, HLB values are calculated using the method of Griffin (Griffin W C; J. Soc. of Cosmetic Chemists, pp. 249-256 (1954)). Thus, as used herein, the “HLB Method” involves a calculation based on the following: HLB=(E+P)/5, where E is the weight percent of oxyethylene content and P is the weight percent of alcohol content (glycerol, sorbitol, etc.). For the compounds herein, glycerol segments with two hydroxyl groups, glycerol segments with one hydroxyl group, and hydroxyl-containing segments of any additional polyhydric molecules were included in the definition of P.

Other methods of calculating the HLB value are available and may be required when determining the HLB value for compounds lacking both E and P groups, as defined above. While the calculated value of HLB may vary depending on the method used, the trends and relative hydrophobicity of materials are expected to be similar.

In one example embodiment, the cationic antimicrobial composition includes a first surfactant with an HLB value of less than about 10, a second surfactant with an HLB value in a range of about 10 to 14, and an optional third surfactant with an HLB value of greater than about 14. The first surfactant can be independently selected from: alkyl esters, alkyl ethers, and alkyl amides, and mixtures and combinations thereof, wherein the alkyl group in any of the alkyl esters, alkyl ethers, and alkyl amides can be independently selected to have 8 to 22 carbon atoms. In some embodiments, the alkyl groups on any of the alkyl esters, alkyl ethers and alkyl amides can include a 1,2 dihydroxy group.

In some embodiments, the first surfactant includes an alkyl ester with an alkyl group having 8 to 22 carbon atoms, which may be a monoester, diester, triester, or a mixture or combination thereof In some embodiments, the alkyl ester is chosen from monoester and diesters, and mixtures and combinations thereof. In some embodiments, the alkyl ester is a monoester.

In some embodiments, surfactants in cationic antimicrobial compositions of the present disclosure include one or more polyethylene glycol (PEG) compounds. Some examples of suitable PEG compounds are described in co-pending U.S. Provisional Application Ser. No. 62/864,187, filed Jun. 20, 2019, entitled “ANTIMICROBIAL SOLUTION”, and incorporated herein by reference.

PEG compounds, also referred to herein as PEGs, together with their derivatives, do not have definite chemical entities, but are compound mixtures having different chain lengths. PEG includes two terminal primary hydroxyl groups that can be used to create mono-, di- and poly-esters, amines, ethers and acetals. PEGs can also create additional compounds and complexes through a reaction in their ether bridges. In the present application the term “PEG compound” refers to PEG derivatives such as, for example, PEG alkyl ethers (e.g., laureths, ceteths, ceteareths, oleths, and PEG ethers of glyceryl cocoates), PEG alkyl esters (e.g., PEG laurates, dilaurates, stearates, and distearates), PEG castor oils, PEG alkyl amides (e.g., PEG cocamines), PEG propylene glycols, PEG 1,2 diols, and other derivates (e.g., PEG soy sterols and PEG beeswax). Since many PEG types are hydrophilic, they are effective penetration enhancers for use in dermatological preparations. The PEG compounds may be used alone or in combination or may be used with optional compounds such as any of alkyl esters, alkyl ethers, and alkyl amides, and mixtures and combinations thereof. Any of the alkyl esters, alkyl ethers and alkyl amides can have an alkyl group independently selected to have 8 to 22 carbon atoms. In some embodiments, the alkyl group can include a 1,2 dihydroxy group.

The cationic antimicrobial composition can include any excipient suitable for pharmaceutical use, that is physiologically well tolerated after administration to the skin and/or a mucosa.

In some embodiments, the PEG compound is a PEG alkyl ester with an alkyl group having 8 to 22 carbon atoms. The PEG alkyl esters, which can also be referred to in the art as PEG fatty acid esters, are the reaction products of a PEG compound (hereafter referred to as a PEG) and a fatty acid.

The PEG in the PEG alkyl ester forms a hydrophilic part of the molecule and the C8-C22 alkyl ester component of the PEG alkyl ester forms a lipophilic part of the molecule. By varying the molecular weight of the PEG and the alkyl ester components of the PEG alkyl ester, surfactant systems covering a wide range of HLB values can be produced. In various embodiments, the PEG alkyl ester compounds in the cationic antimicrobial composition have an HLB value of greater than about 8 and less than about 18, or greater than 8 and less than about 14, or greater than about 10 and less than about 14. In various embodiments, the PEG alkyl ester is a monoester, a diester or a triester, or a mixture or combination thereof. In some embodiments, the PEG alkyl ester is substantially free of triesters, and in some embodiments the PEG alkyl ester is substantially free of both triesters and diesters, and as such consists substantially of monoesters.

In some embodiments, the first surfactant includes a PEG compound chosen from PEG alkyl esters, PEG alkyl ethers, PEG alkyl amides, and mixtures and combinations thereof, wherein the PEG compounds have an alkyl group independently selected to have 8 to 22 carbon atoms.

In some embodiments, the second surfactant with an HLB value in a range of about 10 to about 14 may be included in the cationic antimicrobial composition. In some cases, the second surfactant can improve the solubility of the first low HLB surfactant in a carrier in the cationic antimicrobial composition. Suitable examples of the second surfactant having an HLB in a range of about 10 to about 14 include, but are not limited to, PEG alkyl esters, PEG alkyl ethers, PEG alkyl amides, and mixtures and combinations thereof, wherein the PEG compounds are different from the PEG compounds (if any) in the first surfactant and have independently selected alkyl groups with 8 to 22 carbon atoms.

In some embodiments, an optional third surfactant with an HLB value of greater than about 14 and up to about 18 may be included in the cationic antimicrobial composition. In some cases, the high HLB third surfactant can improve the solubility of a first low HLB surfactant in a carrier in the cationic antimicrobial composition. Suitable examples of the high HLB third surfactant include, but are not limited to, any of alkyl esters, alkyl ethers, and alkyl amides listed above, and mixtures and combinations thereof. In one embodiment, the third surfactant can include PEG hydrogenated castor oils such as those available from BASF, Florham Park, N.J., under the trade designation KOLLIPHOR (HLB=14-16), and the like, non-ionic or zwitter ionic surfactants like TWEEN, and betaines.

In some non-limiting embodiments, alkyl esters that can optionally be utilized as the first and third surfactants in the cationic antimicrobial composition can include any reaction product of a fatty acid and an alcohol.

Suitable alcohols include, but are not limited to, glycerol, 1,2 propane diol, 1,3-propanediol, diacylgalactosylglycerol, diacyldigalactosylglycerol, erythritol, xylitol, adonitol, arabitol, mannitol, sorbitol, polyglycerol, and mixtures and combinations thereof.

Suitable examples of alkyl esters include, but are not limited to, an ester of glycerol with a fatty acid, an ester of propylene glycol with a fatty acid, and mixtures and combinations thereof.

In various embodiments, which are not intended to be limiting, the alkyl esters have an alkyl group with 8 to 22 carbon atoms and are derived from a fatty acid chosen from oleic, linoleic, linolenic, caproic, caprylic, capric, lauric, and mixtures and combinations thereof. In some embodiments, the alkyl ester is derived from caprylic acid, capric acid, and mixtures and combinations thereof. In some embodiments, the alkyl esters include monoglycerides, diglycerides and triglycerides of caprylic acid, monoglycerides, diglycerides and triglycerides of capric acid, and mixtures and combinations thereof. In some embodiments, the fatty acid is chosen from glyceryl mono, di, and tri caprylate, glyceryl monocaprylate and dicaprylate, and glyceryl monocaprylate.

In any of the embodiments above, the alkyl ester can include a mixture of mono, di, and tri esters. In some embodiments, the alkyl ester includes a mixture of monoesters and diesters, and is substantially free of tri esters, or free of tri esters. In some embodiments, the alkyl ester includes monoesters and is substantially free of diesters and triesters, or free of diesters and triesters.

Suitable alkyl esters include, but are not limited to, those available under the trade designation CAPMUL from Abitec, Columbus, Ohio (HLB=6).

In various embodiments, the alkyl esters, alkyl ethers, and alkyl amides are present in the cationic antimicrobial composition at about 1 wt. % to about 10 wt. %, based on the total weight of the composition.

In some embodiments, the cationic antimicrobial composition includes preservatives such as, for example, benzyl alcohol, phenoxy ethanol, and combinations thereof. In one non-limiting example, the alcohol is present in the composition at about 1 wt. % to about 5 wt. %, based on the total weight of the composition.

The cationic antimicrobial composition can optionally include less than about 10 wt. %, or less than about 5 wt. %, or less than about 1 wt. %, or about 0 wt. %, of lower monohydric alcohols, based on the total weight of the composition (±1%). In the present application the term “lower monohydric alcohols” refers to alcohols with a single hydroxyl group and the formula CₙH₂ₙ₊₁OH, wherein n=2 to 5, such as, for example, methanol, ethanol, propanol, isopropyl alcohol, and the like. For example, in some embodiments the cationic antimicrobial composition includes up to about 5 wt. %, or up to about 4 wt. %, or up to about 3 wt. %, of a lower monohydric alcohol such as, for example, isopropanol, which can provide the composition with properties such as enhanced mold resistance.

The reduced amount of C2-C5 monohydric alcohols also provides the cationic antimicrobial composition with good flammability properties when used in a medical or surgical setting, particularly when electrocautery procedures are performed. In some embodiments, for example, the cationic antimicrobial composition has no closed cup flash point at temperatures of 70° F. to 200° F. as measured according to ASTM D-3278-96 e-1.

In various embodiments, the aqueous carrier is present in the cationic antimicrobial composition in an amount of about 5 wt. % to about 98 wt. %, or about 10 wt. % to about 90 wt. %, based on the total weight of the composition (±5%). In various embodiments, the aqueous hydrophilic component includes at least about 80 wt. % water, or at least about 90% water, based on the total weight of the aqueous hydrophilic component (±5%). In some embodiments, the aqueous carrier consists of water, which in this application means that the aqueous carrier is substantially 100% water, or 100% water, based on the total weight of the aqueous carrier (±1%). In some embodiments, the aqueous carrier can optionally include includes predominantly aqueous solutions such as buffers.

In some embodiments, the aqueous carrier in the cationic antimicrobial composition further optionally includes an alcohol chosen from benzyl alcohol, phenoxy ethanol, isopropyl alcohol, ethanol, and mixtures and combinations thereof. In various embodiments, which are not intended to be limiting, the alcohol is present in the cationic antimicrobial composition at about 1 wt. % to about 10 wt. %, based on the total weight of the composition.

In some embodiments, the aqueous carrier in the cationic antimicrobial composition further includes a humectant. As used herein the term “humectant” refers to polar compounds or mixtures of compounds that act to retain or absorb moisture. Suitable humectants include, but are not limited to, polyols, such as glycerin, propylene glycol, dipropylene glycol, polypropylene glycol, glycerine ethoxylates, methyl glucose ethoxylates, polyethylene glycol, polyethylene/polypropylene glycols, and sorbitol. In some embodiments, the humectants include liquid polar solvents such as for example, monoalkyl glycols, glycerol alkyl ethers, monoacyl glycerols, and mixtures and mixtures and combinations thereof. Suitable examples of the liquid polar solvents include, but are not limited to, glycerol, propylene glycol, polyethylene glycol, pentylene glycol, and mixtures and combinations thereof.

Diols such as propylene glycol and pentylene glycol are well tolerated by the skin, and have high affinity to skin and hair. In some embodiments, the diols have a small relatively lipophilic molecular region by virtue of which they may also be considered as somewhat amphiphilic, thus enhancing the solubilization of poorly water-soluble ingredients. In some embodiments, the diols can have substantial antimicrobial properties so that they allow for the formulation of aqueous cationic antimicrobial compositions without any further preservatives, or with reduced preservative levels.

In some embodiments, the aqueous carrier can be a mixture of water and a liquid glycol such as, for example, propylene glycol, pentylene glycol and mixtures thereof. For such mixtures, the ratio of water to glycol (or glycols) may be about 1:10 to about 10:1, or about 1:8 to about 8:1, or about 1:5 to about 5:1. Examples of useful aqueous carriers include water and pentylene glycol (2:1), water and propylene glycol (1:2) In various embodiments, the liquid glycol is present in the cationic antimicrobial composition in any amount of about 0 wt. % to about 50 wt. %, or about 1 wt. % to about 30 wt. %, or about 5 wt. % to about 20 wt. % (±1%), based on the total weight of the cationic antimicrobial composition.

Propylene glycol, in addition to being a humectant, can help increase the solubility of the low HLB surfactant. Certain formulations containing high levels of HLB can have extreme foaming and would be unacceptable for a bathing product. In some formulations, propylene glycol can substantially reduce foaming.

In some embodiments, the optional addition of low levels of stabilizing ingredients in the aqueous carrier can also be advantageous. Salts such as magnesium sulfate may be useful, but the addition of magnesium sulfate can, in some instances, inactivate any bioactive agents, e.g., antimicrobial agents present in the cationic antimicrobial composition such as chlorhexidine gluconate (CHG) in the cationic antimicrobial composition. The addition of water-soluble gums such as guar derivatives, xanthan gum, and thickeners such as hydroxy ethyl cellulose, hydroxy propyl cellulose and carboxyl vinyl polymers may be helpful in stabilizing the cationic antimicrobial composition. Suitable oil phase emulsion stabilizers include, but are not limited to, ethylene/acrylic acid copolymers such as those available under the trade designation AC540 from Allied Signal, Morrison, N.J., and N-vinyl pyrrolidone/olefin copolymers such as that available under the trade designation GANEX V-216 from ISP International Specialty Products, Wayne, N.J.

In another example embodiment, which is not intended to be limiting, the cationic antimicrobial composition includes about 5 wt. % to about 95 wt. % of an aqueous carrier, based on the total weight of the composition; about 1 wt. % to about 10 wt. %, based on the total weight of the composition, of a first surfactant including a first PEG compound such as, for example, a PEG alkyl ester, with an HLB value of greater than about 10 and less than about 14; and about 1 wt. % to about 10 wt. %, based on the total weight of the composition, of a second surfactant, different from the first surfactant, wherein the second surfactant has an HLB value of less than about 10. The second surfactant is chosen from a second PEG compound different from the first PEG compound, an alkyl ester, an alkyl ether, an alkyl amide, and mixtures and combinations thereof, wherein the alkyl esters, ethers and amides have an alkyl group independently selected to have 8 to 22 carbon atoms. In some embodiments, the alkyl group in any of the alkyl esters, alkyl ethers and alkyl amides can include a 1,2 dihydroxy group.

In some embodiments, the cationic antimicrobial composition can optionally include emollient oils such as, for example, silicone fluids, saturated fatty esters and diesters such as diisopropyl adipate, dicapryl adipate, diisopropyl sebacate, dioctyl sebacate, dioctyl ether, glyceryl tricaprylate/caprate, diethyleneglycol dicaprylate/caprate, propylene glycol dipelargonate, polyalkoxylated alcohols such as 15 mole propoxylate of stearyl alcohol, paraffin oils and waxes, animal and vegetable oils including mink oil, coconut oil and derivatives thereof, palm oil, corn oil, cocoa butter, petrolatum, coconut oil, sesame oil, and the like, lanolin derivatives, fatty alcohols such as isostearyl alcohol, isocetyl alcohol, cetyl/stearyl alcohol, and straight chain alcohols from C6-C18 and certain petroleum distillates which are toxicologically safe such as C8-C22 isoparaffin hydrocarbon solvents, e.g., isooctane and isododecane, mixtures of mono, di and tri glycerides of long chain fatty acids, mixtures of propylene glycol mono, di and tri esters of fatty acids. In some embodiments, the same excipients can act as the surfactant component of the composition, depending on how they are formulated (i.e. the remaining excipients).

In some embodiments, the addition of a silicone oil such as dimethicone to the lipophilic component to prepare the microemulsion can also be advantageous in improving the ability of the cationic antimicrobial compositions to act as a barrier to urine, feces, or other indigenous and exogenous materials when used as moisturizing compositions (e.g., moisturizing skin treatments). In some embodiments, the dimethicone may be present at about 1 wt. % to about 5 wt. %, based on the total weight of the composition (±1%). In some embodiments, aloe may be used to help improve the solubility of the dimethicone in the composition to provide further moisturization.

In some embodiments, the cationic antimicrobial composition may optionally include auxiliary emulsifiers conventionally used in cosmetic formulations to ensure stability and extend shelf life of any of the compositions of the present invention. Suitable auxiliary emulsifiers include, but are not limited to, C12-C18 alkyl carboxylic acids such as stearic acid, polypropylene glycol (PPG) (15) stearyl ether (commercially available under the trade designation ARLAMOL E from Uniqema, Wilmington, Del.), and 20-mole ethoxylate of cetyl/stearyl alcohol, polyetherpolyester polymer, such as polyethylene glycol (PEG) (30) polyhydroxy-stearate, MW of approximately 5000 (commercially available under the trade designation ARLACEL P135 from ICI, Wilmington, Del.). In various embodiments, the auxiliary emulsifier is preferably present in an amount of about 1 wt. % to about 20 wt. %, or about 5 wt. % to about 10 wt. %, based on the total weight of the cationic antimicrobial composition.

In various embodiments, the cationic antimicrobial compositions may include further ingredients as required. For example, the cationic antimicrobial compositions may optionally include a further active ingredient, e.g. a corticosteroid, an antibiotic, an antimycotic, and/or an antiviral agent.

The cationic antimicrobial composition may further include up to about 5 wt. %, or up to about 4 wt. %, or up to about 3 wt. %, based on the total weight of the composition, of other optional ingredients including, for example, agents for adjusting the pH (e.g. acids, buffer salts, bases), antioxidants (e.g. ascorbic acid, vitamin E and its derivatives, BHT, BHA, disodium EDTA, etc.), preservatives (e.g. benzyl alcohol, sorbic acid etc.), permeation enhancers (DMSO, diethylene glycol monoethyl ether (DEGEE) available under the trade designation TRANSCUTOL from Gattefossé, Paramus, N.J., menthol, oleic acid, n-alkanols, dimethyl isosorbides, 1-alkyl-2-pyrrolidones, N,N-dimethlyalkanamides, and 1,2-alkanediols, etc.), and the like.

The cationic antimicrobial compositions are stable-for a period of at least about 6 months at room temperature (±1 month). In some embodiments, the cationic antimicrobial composition is stable for a period of about 6 months to about 2 years at room temperature (±1 month). In this application, stable means that the cationic antimicrobial composition is able to kill bacteria in the time kill test after aging. In some cases, even if there is phase separation, the phase-separated product has the ability to kill bacteria in the time kill test when mixed before testing.

In various embodiments, the cationic antimicrobial composition is sufficiently effective against microorganisms on mammalian skin, mucosae, hair, wounds, surgical cavities, and the like, and provides at least a 0.5-log microbial reduction, at least a 1.5-log microbial reduction, at least a 2-log reduction, or at least a 3 log reduction, following 10 min. contact as measured according to ASTM E1874-09.

In some embodiments, the cationic antimicrobial composition is also highly persistent on mammalian skin, mucosae, bone, hair, wounds, surgical cavities, and the like. In this application persistence refers to microbial counts not returning to baseline at a set time, for example 24-hour persistence would be that for 24 hours, the microbial counts have not returned to pre-treatment levels. Efficacy for 24 hours refers to having low bacterial bioburden for a period of 24 hours. A formulation that has high efficacy at 24 hours means that it has very few bacteria left on skin after a period of 24 hours.

In some embodiments, the cationic antimicrobial composition prevents microbial counts from returning to baseline for at least 24 hours, at least 48 hours, or at least 72 hours. In some embodiments, the cationic antimicrobial composition has excellent efficacy for a period of at least 24 hours, at least 48 hours, or at least 72 hours. In some embodiments, the cationic antimicrobial composition has both persistence and high efficacy for a period of at least 24 hours, at least 48 hours, or at least 72 hours.

In various embodiments in which a cationic antiseptic compound is present in the cationic antimicrobial composition, the cationic anionic antimicrobial system reduces the viable bacterial counts by at least 5 log orders within 10 seconds, while the vehicle (antibacterial composition without the cationic antiseptic compound) reduce viable bacterial counts by less than 0.5 log orders within 10 seconds.

The cationic antimicrobial composition can be easily manufactured and scaled up into industrial scale production. The cationic antimicrobial composition can be formed as the ingredients are combined and mixed together, even in the absence of high shear conditions or pressure homogenization. Therefore, the cationic antimicrobial composition may be prepared using any standard mixing equipment which is suitable for the preparation of liquid pharmaceutical formulations at the appropriate scale. Optionally, ultrasound treatment of the combined ingredients may be used to accelerate formation.

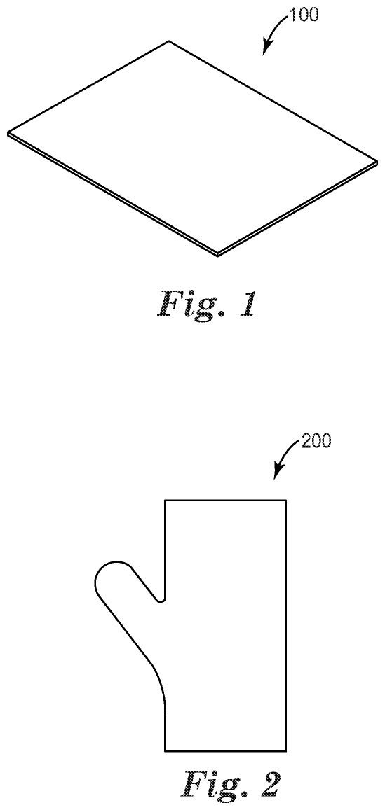

Pre-loaded wipes of the present disclosure (for example, 100 in FIG. 1) include a plurality of fibers. Fibrous wipes are well-known, and can include, for example, flocked materials, nonwoven materials, knit materials, and woven materials. In a number of instances, the fibers in wipes have also incorporated various textured surfaces to facilitate cleaning ability. The fibers are typically selected to have a good balance of properties, including moisture absorption and/or scrubbing capability.

Fibers have many physical characteristics including, for example, linear mass density, expressed as “denier” (abbreviated as “D”). Denier is a unit of measure for the linear mass density of fibers and is defined as the mass in grams per 9000 meters of the fiber. Denier is used widely in the art of textiles in reference to the fineness of fibers in fabrics. Microfibers are those fibers having a denier value (“D”) of less than 1.0. Nanofibers are those fibers having a much lower denier value, as low as 0.01, or even less.

For fibers having similar materials, cross-sectional profile and surface characteristics, microfibers (i.e., fibers having a denier value less than 1.0) have a higher surface area than comparable fibers having a denier value greater than 1.0. The higher surface area can enhance absorption characteristics of the fibers, which is helpful, for example, in loading antimicrobial solutions into wipes having fibers with denier less than 1.0. On the other hand, this same higher surface area can sometimes result in a diminished distribution of antimicrobial compositions from the wipes, or at least certain components in the antimicrobial solutions. Some antiseptic compounds in the antimicrobial compositions may be retained in the wipes at higher levels than comparable wipes having larger denier fibers. Over time, a microfiber wipe pre-loaded with an antimicrobial solution may exhibit an increasing retention of the antimicrobial composition, or at least some components in that composition.

Wipes comprising fibers less than 1 denier loaded with a cationic antiseptic compound were highly effective at reducing the bioburden in a contaminated site. Without being bound by theory, it is thought that for wipes comprising fibers less than 1 denier, these very small fibers can access deep layers of treatment sites (for example, skin), thereby providing an effective distribution of antimicrobial compositions, into those deeper layers of skin treatment sites, and an effective kill of pathogens at those layers. The microfibers are thought to have increased mechanical forces at the interface of the wipe and the treatment site, due to a large contact area, for help in delivering antimicrobial compositions.

The materials of the fibers in wipes of the present disclosure are generally selected from among polyesters, polyamides, polypropylene, polyphenylene sulfide, and various mixtures and combinations of those materials. In some preferred embodiments, the fiber material is a polyester, for example, polyethylene terephthalate (PET). The wipe materials are selected to be capable of being impregnated with the antimicrobial composition.

Fibers with denier less than 1 of the present disclosure can be selected to have various morphologies. A cross-sectional profile of the microfiber, taken perpendicular to the length of the microfiber can be solid (including monolithic, or multi-component solid fibers having, for example, “island in sea” distribution of components), or include a cavity running the length of the fiber, or have various kinds of split fiber geometries (see, for example WO2007/0782030), all well-known in the art.

The exterior surface of the fibers can have a circular or non-circular cross-sectional geometry, and if there is an interior cavity (or plurality of interior cavities), the interior surface(s) can likewise have a circular or non-circular cross-sectional geometry. It will be evident that there is a wide selection of fiber characteristics.

Some non-limiting examples of useful wipes having fibers with denier less than 1 include the 3M SCOTCH-BRITE KITCHEN CLOTH® (from 3M Co., St. Paul, Minn.), and the stitch-bonded PURECLEAN cleaning cloth (from INTEX DIY, Inc., Villa Rica, Ga.).

Wipes of the present disclosure preferably have a thickness in a range from about 1.5 mm to about 3.0 mm, with a thickness of about 2.3 mm being more preferable. The basis weight of the wipes can be in a range of from about 15 g/m² to about 250 g/m². The wipes are suitably soft to permit wiping of skin without breaking skin at the treatment site.

In some embodiments, the wipe is mitt-shaped (for example, 200 in FIG. 2) and dimensioned to receive a hand of the user. This can provide a convenient means for the user to wipe across a treatment site. In some embodiments, the mitt-shaped wipe includes a barrier layer (e.g., a flexible polymeric layer) between the antimicrobial composition in the pre-loaded wipe and the hand of the user. However, the antimicrobial composition is generally formulated so as to not be irritating to skin.

The wipe can be pre-loaded with the cationic antimicrobial composition using any conventional methods, for example, pouring the cationic antimicrobial composition onto the wipe, or dipping the wipe into a bath of the cationic antimicrobial composition. The resulting pre-loaded wipe may then be packaged in a sealed environment (individually or with multiple pre-loaded wipes) for ease of handling and to prevent evaporation of components in the antimicrobial composition.

The present disclosure includes a system for disinfecting a treatment site chosen from skin, hair, mucosae, wounds, and body cavities, the system including a cationic antimicrobial composition distributed throughout a wipe. The wipe includes a plurality of fibers having a denier value of less than about 1.0, and the cationic antimicrobial composition is distributed from the wipe to the treatment site, in an amount effective to kill a microorganism chosen from bacteria, fungi, viruses, and mixtures and combinations thereof. In some embodiments, the cationic antimicrobial composition includes a cationic antiseptic compound chosen from bispyridines, biguanides, bisbiguanides, polymeric biguanides, salts thereof, and mixtures and combinations thereof. In some embodiments, the cationic antiseptic compound is chosen from octenidine dihydrochloride, polyhexamethylene biguanide (PHMB), and chlorhexidine gluconate (CHG), and combinations thereof. In some embodiments of the system, the antiseptic compound includes CHG. In some embodiments of the system, the cationic antiseptic compound is CHG.

In some embodiments of the system, the cationic antimicrobial composition further includes a surfactant system chosen from a first surfactant having an HLB value of less than about 10, a second surfactant different from the first surfactant and having an HLB value in a range from about 10 to about 14, a third surfactant different from both the first surfactant and the second surfactant and having an HLB value in a range from about 14 to about 18, and combinations thereof.

The present disclosure includes a method of disinfecting a treatment site chosen from skin, hair, mucosae, wounds, and body cavities. The method includes distributing a cationic antimicrobial composition from a pre-loaded wipe to the treatment site. The pre-loaded wipe includes the cationic antimicrobial composition distributed throughout a plurality of fibers having a denier value of less than about 1.0. In the method, the cationic antiseptic compound is distributed from the pre-loaded wipe to the treatment site in an amount effective to kill a microorganism chosen from bacteria, fungi, viruses, and mixtures and combinations thereof.

In some embodiments of the method, the cationic antimicrobial composition includes a cationic antiseptic compound chosen from bispyridines, biguanides, bisbiguanides, polymeric biguanides, salts thereof, and mixtures and combinations thereof. In some embodiments, the cationic antiseptic compound is chosen from octenidine dihydrochloride, polyhexamethylene biguanide (PHMB), and chlorhexidine gluconate (CHG), and combinations thereof. In some embodiments of the method, the antiseptic compound includes CHG. In some embodiments of the method, the cationic antiseptic compound is CHG.

In some embodiments of the method, the cationic antimicrobial composition further includes a surfactant system chosen from a first surfactant having an HLB value of less than about 10, a second surfactant different from the first surfactant and having an HLB value in a range from about 10 to about 14, a third surfactant different from both the first surfactant and the second surfactant and having an HLB value in a range from about 14 to about 18, and combinations thereof.

In some embodiments, the method further includes wiping the pre-loaded wipe over the treatment site. The wiping is typically done gently enough so as to not damage the treatment site by, for example, breaking skin at the treatment site.

In some embodiments of the method, the treatment site is porous, and the method further includes penetrating the cationic antimicrobial composition into the porous treatment site.

In some embodiments, the method further includes releasing the cationic antiseptic compound is from the pre-loaded wipe to the treatment site in an amount of at least 50 wt. % or at least 60 wt. %, or at least 70 wt. %, or at least 80 wt. %, of at least 90 wt. %, based on the total weight of cationic antiseptic compound in the antimicrobial solution.

Embodiments of the invention will now be illustrated with reference to the following non-limiting examples.

### EXAMPLES

TABLE 1
Materials

| Designation | Description | Source |
|---|---|---|
| CHG | Chlorhexidine gluconate | Medichem, |
| | | Germany |
| MF-1 | A microfiber cloth available | 3M Co., St. Paul, |
| | under the trade designation “3M | MN |
| | SCOTCH-BRITE KITCHEN | |
| | CLOTH ®” | |
| MF-2 | A microfiber cloth having | Tietex Intl., |
| | microfibers stitched into a | Spartanburg, SC |
| | nonwoven backing, available | |
| | under the trade designation | |
| | “TIETEX T768” | |
| NW-1 | A nonwoven material having | Welcron, Seoul, |
| | fibers with a denier value of | KR |
| | 1.5, the material available under | |
| | the trade designation | |
| | “WELCRON N120P” | |
| CAPMUL 808G | Glyceryl monocaprylate, | |
| | available under the trade | |
| | designation “CAPMUL 808G” | |
| 2-Phenoxyethanol | 2-Phenoxyethanol | Sigma-Aldrich, |
| | | St. Louis, MO |
| Propylene glycol | Propylene glycol | Sigma-Aldrich, |
| | | St. Louis, MO |
| Gluconolactone | — | Sigma-Aldrich, |
| | | St. Louis, MO |
| LABROSOL | Caprylocaproyl Polyoxyl-8 | Gattefossé, Lyon, |
| | glycerides | France |
| KOLLIFOR RH | (According to manufacturer) a | Sigma-Aldrich, |
| 40 | nonionic surfactant, including | St. Louis, MO |
| | glycerol polyethylene glycol | |
| | hydroxy- stearate, fatty acid | |
| | glycerol polyglycol esters, other | |
| | polyethylene glycols and | |
| | glycerol ethoxylate, available | |
| | under the trade designation | |
| | “KOLLIFOR RH 40” | |
| LABRAFIL 1947 | Oleoyl polyoxyl-6 glycerides, | Gattefossé, Lyon, |
| | available under the trade | France |
| | designation “LABRAFIL 1947” | |
| PEG-12 glyceryl | — | Parchem, New |
| laurate | | Rochelle, NY |
| Propylene glycol | — | Sigma-Aldrich |
| | | St. Louis, MO |
| IPA | Isopropyl alcohol | Sigma-Aldrich, |
| | | St. Louis, MO |

### Test Method for Antimicrobial Efficacy

Samples of wipe materials were wetted (pre-loaded) with 60-70 wt. % of their maximum capacity of liquid. A 5 inches by 5 inches (approximately 13 cm by 13 cm) area of pig skin was wiped in a back-and-forth motion for 1.5 min, then the wipe material was moved or folded to create a new surface and the area of pig skin was wiped for an additional 1.5 min. Microbial colony forming units (CFU) were detected using the cup scrub method (ASTM E1874) for 3 times per skin outside of treated areas (baselines) and once in each treated area, 10 minutes after treatment. Log reduction was determined by subtracting the log of CFU/cm² in a treated area from an averaged baseline of nontreated areas.

### Example 1: Release of Chlorhexidine Gluconate (CHG) from Wipe Materials

With reference to Table 2, the listed wipe material was saturated to 100% capacity with a solution of 2 wt. % CHG in water, and lightly squeezed to removed excess liquid. Each wipe sample was stored in a plastic zip-lock bag for the indicated time, then the liquid was collected by wringing out the wipe material. A 10 microliters sample of the collected liquid was diluted in 1 mL of deionized water, mixed, and then diluted another 10-fold. UV absorbance at 250 nm wavelength was measured and compared to a standard curve to determine concentration of CHG in the sample, with results as summarized in Table 2.

TABLE 2
CHG percent release from wipe materials
after indicated time period

| | Time | | |
|---|---|---|---|
| Wipe | 1 h | 24 h | 72 h |
| MF-1 | 93% | 90% | 89% |
| MF-2 | 93% | 93% | 95% |
| NW-1 (1.5 D) | 97% | 94% | 90% |

Based on the CHG % release values in Table 2, loading volumes in wipe materials of the following examples were normalized to release approximately equal amounts of antimicrobial formulation from the wipe materials. Loading volumes were adjusted by saturating the wipe and then expressing a weighed amount to achieve the target loading volume.

### Example 2: Antimicrobial Efficacy of Wipes Having CHG Formulation

With reference to Table 3, a formulation of CHG with other components in water was prepared. Wipe materials were wetted with the CHG formulation or saline and tested on porcine skin according to the Test Method for Antimicrobial Efficacy, with log reduction results as summarized in Table 4.

TABLE 3
CHG formulation in water

| | Weight percent (wt. %) in |
|---|---|
| Formulation component | water solution |
| Chlorhexidine gluconate (CHG) | 2 |
| CAPMUL 808G | 5 |
| 2-Phenoxyethanol | 1 |
| Propylene glycol | 10 |
| Gluconolactone | 0.2 |
| Isopropanol (IPA) | 4 |

TABLE 4

| | CHG Formulation, log | Saline, log reduction |
|---|---|---|
| Wipe | reduction (n > 10) | (n > 11) |
| MF-1 | 3.0 | 1.4 |
| NW-1 (1.5 D) | 1.9 | 1.1 |

### Example 3: Efficacy of Bacterial Reduction in Combinations of Wipe Materials and CHG Formulations

With reference to Table 5, Formulations A and B with CHG and other components in water were prepared, along with a 2 wt. % solution of CHG in water. The wipe materials listed in Table 6 were wetted with the CHG formulation and tested on porcine skin according to the Test Method for Antimicrobial Efficacy, with log reduction results as summarized in Table 6.

TABLE 5

| | Formulation A, | Formulation B, |
|---|---|---|
| Formula component | wt. % in water | wt. % in water |
| CHG | 2 | 2 |
| LABRASOL | 0.5 | 0 |
| LABRAFIL 1947 | 0.5 | 0 |
| KOLLIPHIR RH 40 | 1.5 | 0 |
| CAPMUL 808G | 0 | 1 |
| PEG-12 glyceryl laurate | 0 | 2 |
| 2-Phenoxyethanol | 1 | 1 |
| Propylene glycol | 10 | 10 |
| IPA | 4 | 4 |

TABLE 6

| | CHG, 2 wt. % in water, | Formulation A, | Formulation B, |
|---|---|---|---|
| | log reduction | log reduction | log reduction |
| Wipe | (n > 10) | (n > 10) | (n > 10) |
| MF-1 | ND | ND | 2.3 |
| MF-2 | 2.4 | 2.4 | 2.3 |

In Table 6, “ND” = not determined

### Example 4: Comparison of Efficacy for a Microfiber Wipe Vs. A Larger Denier Nonwoven Wipe

With reference to Table 7, It was observed that the wipe with microfibers had better efficacy than a comparable wipe with a larger denier non-woven.

TABLE 7

| | CHG 2 wt. % in water, | Saline, log reduction |
|---|---|---|
| Wipe | log reduction (n > 10) | (n > 11) |
| MF-2 | 2.4 | 1.4 |
| NW-1 (1.5 D) | 1.7 | 1.1 |

### Example 5: Effect of Skincare Excipients on Efficacy

With reference to Table 8, Formulations D and E of CHG with other components in water were prepared. Wipe materials were wetted with either Formulation D or Formulation E and tested on porcine skin according to the Test Method for Antimicrobial Efficacy, with log reduction results as summarized in Table 9.

TABLE 8

| | Formulation D, | Formulation E, |
|---|---|---|
| Formula component | wt. % in water | wt. % in water |
| CHG | 2 | 2 |
| LABRASOL | 0 | 2 |
| CAPMUL 808G | 1 | 0 |
| PEG-12 glyceryl laurate | 2 | 0 |
| 2-Phenoxyethanol | 1 | 0 |
| Propylene glycol | 10 | 10 |
| IPA | 4 | 4 |

TABLE 9

| | Formulation D, | Formulation E, | Saline, log |
|---|---|---|---|
| | log reduction | log reduction | reduction |
| Wipe | (n > 10) | (n > 10) | (n > 10) |
| MF-1 | 1.9 | ND | ND |
| MF-2 | 2.7 | ND | 1.4 |
| NW-1 (1.5 D) | 1.6 | 2.0 | 1.1 |

In Table 11, “ND” = not determined

1. A pre-loaded wipe comprising:
a plurality of fibers having a denier value of less than about 1.0; and
an antimicrobial composition comprising a cationic antiseptic compound selected from a bispyridine, a biguanide, a bisbiguanide, a polymeric biguanide, a salt thereof, and a combinations thereof.
wherein the cationic antimicrobial composition is distributed throughout the plurality of fibers.

2. The pre-loaded wipe of claim 1, wherein the cationic antiseptic compound is selected from octenidine dihydrochloride, polyhexamethylene biguanide (PHMB), chlorhexidine gluconate (CHG), and a combination thereof.

3. The pre-loaded wipe of claim 1, the antimicrobial composition further comprising a first surfactant characterized by an HLB value of less than about 10,
wherein the first surfactant is selected from a polyethylene glycol (PEG) alkyl ester, an alkyl ester, an alkyl ether, an alkyl amide and mixtures and a combinations thereof,
wherein the first surfactant comprises an alkyl group having 8 to 22 carbon atoms.

4. The pre-loaded wipe as claimed in claim 3, the cationic antimicrobial composition further comprising a second surfactant characterized by an HLB value of about 10 to about 14,
wherein the second surfactant is selected from a polyethylene glycol (PEG) alkyl ester,
wherein the second surfactant is different from the first surfactant,
wherein the second surfactant comprises an alkyl group having 17 to 21 carbon atoms.

5. The pre-loaded wipe of claim 4, the cationic antimicrobial composition further comprising a third surfactant characterized by an HLB value in a range from about 14 to about 18, wherein the third surfactant is a PEG compound chosen from PEG alkyl esters, PEG alkyl ethers, PEG alkyl amides, and mixtures and combinations thereof, and wherein the third surfactant is different from the first surfactant.

6. The pre-loaded wipe of claim 1, the cationic antimicrobial composition further comprising a surfactant system, the surfactant system comprising a PEG alkyl ester having an HLB value in a range from about 8 to about 14; and wherein the PEG alkyl ester is a reaction product of a PEG and a C8 to C22 fatty acid.

7. The pre-loaded wipe of claim 1, wherein at least 50 wt. % of the cationic antiseptic compound is released from the plurality of fibers upon wiping the pre-loaded wipe over a treatment site chosen from skin, hair, mucosae, wounds, and body cavities.

8-11. (canceled)

12. A method of disinfecting a treatment site chosen from skin, hair, mucosae, wounds, and body cavities, the method comprising:
providing a pre-loaded wipe of claim 1; and
contacting the pre-loaded wipe to the treatment site such that the cationic antimicrobial composition from the pre-loaded wipe is distributed to the treatment site in an amount effective to kill a microorganism chosen from bacteria, fungi, viruses, and mixtures and combinations thereof.

13. The method of claim 12, wherein the cationic antiseptic compound is chosen from bispyridines, biguanides, bisbiguanides, polymeric biguanides, salts thereof, and mixtures and combinations thereof.

14. The method of claim 13, wherein the cationic antiseptic is chosen from octenidine dihydrochloride, polyhexamethylene biguanide (PHMB), and chlorhexidine gluconate (CHG), and combinations thereof.

15. The method of claim 12, the cationic antimicrobial composition further comprising a surfactant system, the surfactant system comprising a surfactant selected from
a first surfactant characterized by an HLB value of less than about 10,
a second surfactant different from the first surfactant, the second surfactant characterized by an HLB value in a range from about 10 to about 14,
a third surfactant different from both the first surfactant and the second surfactant, the third surfactant characterized by an HLB value in a range from about 14 to about 18, and
a combination thereof.

16. The method of claim 12, further comprising:
wiping the pre-loaded wipe over the treater site.

17. The method of claim 12, wherein the treatment site is porous and the method further comprising:
penetrating the cationic antimicrobial composition into the porous treatment site.

18. The method of claim 12, releasing at least 50 wt. % of the cationic antiseptic compound from the pre-loaded wipe to the treatment site.

19. A pre-loaded wipe comprising:
a plurality of fibers having a denier value of less than about 1.0; and
an antimicrobial composition comprising:
a cationic antiseptic compound selected from a bispyridine, a biguanide, a bisbiguanide, a polymeric biguanide, a salt thereof, and a combination thereof, and
a surfactant system comprising a first surfactant characterized by an HLB value of less than about 10,
wherein the cationic antimicrobial composition is distributed throughout the plurality of fibers.

20. The pre-loaded wipe of claim 19, further comprising:
a second surfactant different from the first surfactant, the second surfactant characterized by an HLB value in a range from about 10 to about 14.

21. The pre-loaded wipe of claim 19, further comprising:
a second surfactant different from the first surfactant, the second surfactant characterized by an HLB value in a range from about 10 to about 14, and
a third surfactant different from both the first surfactant and the second surfactant, the third surfactant characterized by an HLB value in a range from about 14 to about 18.
